(19) United States
(12) Patent Application Publication
(10) Pub. No.: US 2016/0270488 A1
(43) Pub. Date: Sep. 22, 2016

(54) LAMINATE FOR A HOOK-AND-LOOP-FASTENER, AND METHOD OF MAKING A LAMINATE FOR A HOOK-AND-LOOP FASTENER

(71) Applicant: MONDI Gronau GmbH, Gronau (DE)

(72) Inventors: Dieter HOMOELLE, Ochtrup (DE); Georg BALDAUF, Laer (DE)

(21) Appl. No.: 14/662,949

(22) Filed: Mar. 19, 2015

(51) Int. Cl. A44B 18/00; A61F 13/62

(52) U.S. Cl. CPC A44B 18/0069; A61F 13/627; A44B 18/0023

(57) ABSTRACT

A laminate for a hook-and-loop fastener is made by supplying a film as a substrate and supplying a textile web that has a base structure knitted from threads and has loop threads incorporated into the base structure by knitting. The loop threads form loops provided for attachment to hook-and-loop hooks and formed from a textured multifilament yarn. The substrate and the textile web are bonded together to form a material web by an adhesive arrayed in a pattern of adhesive surfaces and adhesive-free regions. Individual pieces of the laminate are then separated from the material web.

The invention relates to a laminate for a hook-and-loop-fastener, in particular for a diaper fastener, comprising a substrate and a textile web that is laminated to the substrate and that has a base structure made of machine-knitted threads and looped threads that are incorporated into the base structure by knitting and form loops provided for attachment to hook-and-loop hooks. The invention further relates to a method of making a laminate for a hook-and-loop-fastener.

Hook-and-loop fasteners, in their standard design, consist of a loop part and a hook part. Corresponding hook-and-loop fasteners are also referred to as hook-and-loop systems. Hook-and-loop fasteners are used for both reusable articles and disposable articles such as diapers, particularly baby diapers or incontinence products for adults.

In the case of diaper fasteners for disposable diapers, in practice primarily laminate elements made of a printed polyethylene film are used as the substrate, onto which a knitted fabric made of polyamide as the loop film is adhesively laminated. The laminate can be installed, for example, in the waist area at the level of the abdomen or lower abdomen of a user, so that it can receive two hook patches that wrap around the waist at the sides. In this context, the laminated element is sometimes referred to in practical use as the “landing zone.”

With disposable products such as diapers in particular, low production costs along with low basis weights of the substrate and the textile web laminated thereto are generally sought, as they give the textile web designed as a knitted product an open and airy structure.

To achieve both low production costs and a strong hook-and-loop action, the substrate and the textile web can be attached to one another in a pattern of adhesive surfaces and adhesive-free regions. Correspondingly designed generic laminates are known from EP 1 579 779 [U.S. Pat. No. 7,527,848] and from EP 1 690 967 [U.S. Pat. No. 7,670,662], with the adhesive according to EP 1 690 967 being arranged as a frame around the periphery of the individual laminate patch, in order to prevent tearing off in this area.

The laminates known from the prior art can be produced with a low basis weight and therefore cost effectively. However, the laminates have the disadvantage that the open structure of the knitted product with the substrate film beneath it has an undesirable, less soft look and feel.

Prior-art laminates are known having a surface made of nonwoven that forms loops for anchoring hook-and-loop hooks. However, to achieve sufficient hooking action and strength, a relatively heavy basis weight of typically 40 to 50 g/m² is necessary. A pleasing surface with a soft touch effect is thus associated with relatively high production costs, due to the greater basis weight. A laminate having a nonwoven provided for attachment to hook-and-loop hooks is known from WO 2003/105621 [US 2006/0217022].

Laminates having a surface made of nonwoven are likewise known from EP 2 191 801 [U.S. Pat. No. 8,347,466], from WO 2000/042964 [U.S. Pat. No. 6,647,600] and from US 2004/022993 [U.S. Pat. No. 7,294,387], in which additional loops are worked into the nonwoven. Since a nonwoven material is used as the textile base structure, the entire laminate has a soft, pleasing look; however, the use of nonwoven results in a relatively heavy basis weight and therefore high production costs.

The object of the present invention is to provide a laminate having loops formed by knitting that has a soft surface at a low basis weight. The object is further to specify a method of making such a laminate.

The subject matter of the invention and the solution to the problem are a laminate according to claim 1 and a method according to claim 13.

To achieve the easiest possible production at a low basis weight, it is provided within the scope of the invention that the base structure is knitted. In particular, the base structure can be knitted from threads of a first base yarn and from threads of a second base yarn. Proceeding therefrom, the first base yarn and the second base yarn are preferably made of polyamide, for example PA6 or PA66. Proceeding from a laminate having a knitted base structure and the features described in the introductory part, according to the invention the loop threads are made of a textured multifilament yarn, in particular a polyester- or polyamide-based multifilament yarn.

By using a textured multifilament yarn for the loop threads, a particularly soft, less open surface is achieved that is characterized by the look of a smooth, textile-like layer and a softer feel. In particular, a look with a soft feel can be achieved that corresponds approximately to the look of the outer layer of standard diapers, referred to as the “backsheet.”

In contrast to prior-art laminates that have a textile web formed entirely by knitting, a lattice structure, formed by the hook-and-loop strands extending in the direction of production and the rows of stitches extending transversely thereto, is less apparent, even with a low basis weight.

The textured multifilament yarns better fill in the open interstices in the loose, open textile web. As a result, the laminate has a high-quality appearance to the user.

Even if the laminate for a hook-and-loop fastener is generally arranged such that, under normal use, it does not come into contact with the skin of a user, such contact cannot always be prevented in practice. The softer feel of the embodiment according to the invention therefore also results in reduced risk of skin irritation or similar impairment to a user.

Through texturing, the multifilament yarn, which is smooth at first, is given a wavy, crimped or even shirred structure during yarn production, depending on the degree of texturing. The polyester- or polyamide-based multifilament yarn is therefore given a structure that is more typical of natural fibers.

In principle, various methods may be used for the shirring or texturing. The individual filaments of the multifilament yarn can be shaped purely mechanically by a blowing process and/or by rolling, or by a combination of mechanical and thermal processes. In terms of the present invention, intermingling and twisting by a fluid stream, for example using air jets, is particularly suitable for achieving a fine, soft texturing of the multifilament yarn for the loop threads.

Various polymers, such as polyester, polyamide, polyactide and polypropylene, along with polymer mixtures, are suitable for the textured multifilament yarn of the loop threads.

The textured multifilament yarn is particularly preferably produced with a polyethylene terephthalate (PET) base that is characterized by good mechanical properties and can be readily textured.

It is also advantageous for the multifilament yarn to have a higher number of filaments than known laminates with loops worked in. It is thus provided according to a preferred embodiment of the invention that the textured multifilament yarn of the loop threads has between 11 and 25, in particular between 13 and 20 textured filaments with a linear density of between 35 dtex and 55 dtex.

According to the invention, it is provided that textured multifilament yarn is used for the loop threads in producing the textile web. It can also optionally be provided that the loops are processed mechanically even after the textile web has been laminated to the substrate, in order to align the loops, to further separate the individual filaments from one another, or to produce an even more intense twisting or shirring in the individual filaments. For this purpose, the produced composite material can also be processed using an air jet or brushes, for example.

Within the scope of the invention, the base structure may be made of a nonwoven material, in which case the loop threads are worked into the nonwoven base structure. In this embodiment, a particularly smooth, soft structure results in which ultimately the lattice-like structure formed by warp strands (wale) and rows of stitches that is typical of a fully knitted textile web is not or is barely discernible.

The base structure is provided essentially for receiving and holding the loop threads. The base structure can be formed, for example, from monofilament threads that typically have a linear density of between 15 and 30 dtex, for example 22 dtex.

Furthermore, the base structure can also influence the characteristics of look and feel, at least to a certain extent.

According to one variant of the invention, the first base yarn is made of a monofilament yarn having a linear density of between 15 and 30 dtex and the second base yarn is made of a preferably untextured multifilament yarn having a linear density of between 35 and 55 dtex.

The linear density of the textured multifilament yarn for the loop threads and that of the first base yarn and the second base yarn is chosen based on the mechanical requirements and the requirements in terms of look and feel for the laminate. Furthermore, the hook-and-loop characteristics are determined substantially by the properties of the yarns and the density of the textile web that is produced. With respect to the use of the material as part of a hook-and-loop fastener for a disposable article, opposing issues must be taken into consideration. On one hand, the lowest possible basis weight of the textile web is desirable for reasons of cost. On the other hand, the textile web must also have sufficient strength and sufficient capacity for attachment to hook-and-loop hooks.

In light of these requirements, the textile web preferably has a basis weight of between 15 and 45 g/m², preferably between 18 and 25 g/m².

The described textile web by itself also has an inventive advantage. The invention therefore also relates to the textile web as described, without the substrate. That is, a textile web having a base structure formed from threads or fibers and having loop threads worked into the base structure by knitting that form loops provided for attachment to hook-and-loop hooks, the loop threads being made of a textured multifilament yarn.

Within the scope of the invention, in addition to a customary film as the substrate, a nonwoven material is also generally possible, and the substrate is frequently printed with a decorative pattern that is then visible through the laminated textile web. If the substrate according to a preferred embodiment of the invention is formed from a single-layer or multilayer film, at least the surface of the film, which is adhesively fastened to the textile web, has a polyolefin, in particular polyethylene, as its main constituent. For reasons of cost, a polyethylene monofilm is particularly suitable, with the thickness of the film—regardless of its specific embodiment—preferably being between 8 μm and 30 μm.

When a film is used as the substrate, a decorative printed pattern can be easily printed on the film. The decorative printed pattern is preferably on the face opposite the textile web and is then visible through the film and the textile web that is on the outside when the material is on a diaper.

The substrate and the textile web are preferably attached to one another by an adhesive. The adhesive can be arrayed in a pattern rather than covering the entire surface, with the pattern having adhesive surfaces and adhesive-free regions. In addition, depending on the form of the laminate, a strip of adhesive forming a circumferential frame can also be provided, with corresponding embodiments being described in EP 1 690 967. The area coverage is preferably approximately 20%. For example, the adhesive can be formed in a pattern of intersecting, wave-shaped, overlapping adhesive strips, with such a pattern being referred to as a “dog bone” pattern. A corresponding pattern is known, for example, from FIG. 2a of EP 2 439 323 [US 2012/0088061].

The application weight of the adhesive (in relation to the total area of adhesive surfaces and adhesive-free regions) is preferably approximately 2 g/m².

A further feature of the invention is a method of making a laminate for a hook-and-loop-fastener, where a film is supplied as a substrate, a textile web is supplied that has a base structure knitted from threads and loop threads worked into the base structure by knitting and forming loops provided for attachment to hook-and-loop hooks and being formed from a textured multifilament yarn, in particular a polyester- or polyamide-based multifilament yarn, the substrate and the textile web are connected to form a material web by an adhesive, arrayed in a pattern of adhesive surfaces and adhesive-free regions, and individual pieces of the laminate are separated from the material web.

In addition to using textured multifilament yarn for the loop threads, once the material web has been produced, the textile web can also optionally be bulked up further using compressed air, brushes or some other mechanical treatment.

Pieces of the laminate are separated from the material web and are each arranged on a waist region of a diaper.

The substrate in the form of a film is also printed.

In the following, the invention will be specified in greater detail within the context of one embodiment example, with reference to the figures. In the drawings:

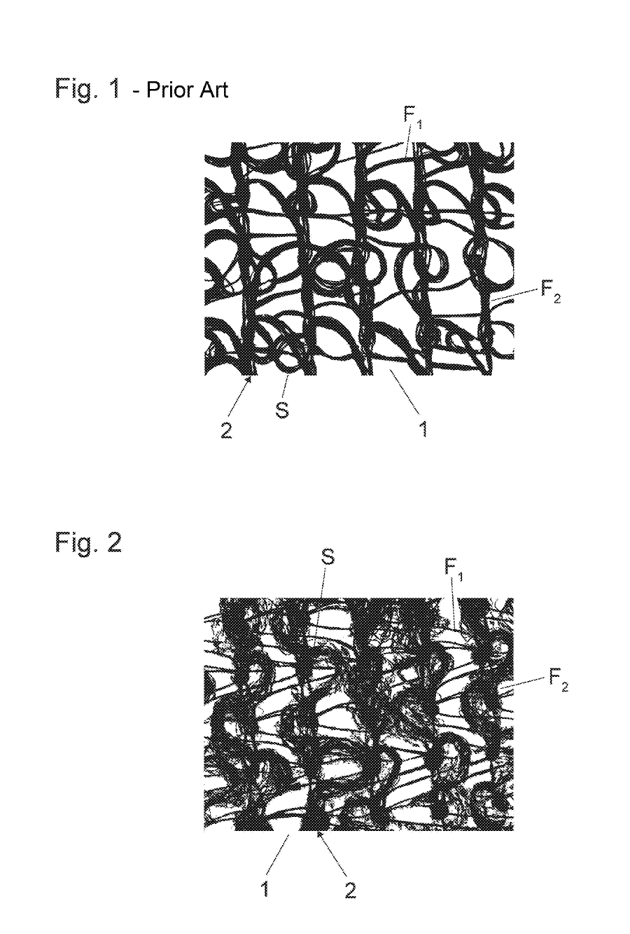

FIG. 1 shows a prior-art laminate,

FIG. 2 shows a laminate according to the invention.

FIG. 1 shows a known laminate for a hook-and-loop-fastener, in particular for a diaper fastener, having a substrate 1 in the form of a film, and a textile web 2 laminated onto the substrate 1. The textile web 2 is knitted and has a knitted base structure comprising threads F₁ of a first base yarn and threads F₂ of a second base yarn.

Loop threads S are also incorporated into the base structure by knitting, and form loops for attachment to hook-and-loop hooks.

The substrate 1 and the textile web 2 are connected by an unillustrated adhesive that is arrayed in a pattern formed adhesive surfaces and adhesive-free regions. Suitable for this is a “dog bone” structure known from FIG. 2a of EP 2 439 323, for example, with intersecting, wave-shaped adhesive strips, where the surface coverage of the adhesive surfaces is preferably approximately 20% and the average application weight over the entire surface is 2 g/m². Suitable adhesives include monocomponent polyurethane-based adhesives that particularly harden in humidity and/or when sprinkled with water.

In the embodiment example of FIG. 1 according to the prior art, the threads F₁ and F₂ of the first and the second base yarn are made of untextured PA6 monofilaments having a linear density of 22 dtex, with a untextured PA6 multifilament yarn having nine filaments being provided for the loop threads.

In contrast, FIG. 2 shows a laminate for a hook-and-loop fastener according to the invention, where the loop threads S are made of a textured multifilament yarn. It is clear that the individual filaments of loop threads S are highly shirred and bulked up, resulting in a particularly smooth, soft look and feel. The lattice-like structure with warp strands (wale) and rows of stitches extending transverse thereto, which is clearly visible in FIG. 1, is less discernible.

Within the scope of the embodiment, the loop threads are made of polyethylene terephthalate (PET) having a linear density of 44 dtex, with the textured multifilament yarn having 13 filaments.

Whereas the first base yarn, as before, is made of a thread F₁ in the form of a monofilament yarn having a linear density of 22 dtex, the thread F₂ of the second base yarn is a untextured monofilament yarn having a linear density of 44 dtex. Threads F₁ and F₂ of the first base yarn and the second base yarn are made of PA6, and therefore in the above-described embodiment, the textile web of the laminate according to the invention is not homogeneous. In principle, however, it is also possible within the scope of the invention for loop threads S to be made of a textured polyamide multifilament yarn or of textured multifilament yarn having a different polymer base.

Proceeding from a laminate according to FIG. 2, in order to verify the functional characteristics of the material element by modifying the mesh size of the textile web 2 and varying the linear density accordingly, the basis weight was varied within a range of 18 to 35 g/m². A functional diaper fastener with satisfactory peel and shear values was achieved across the entire range.

1. In a laminate for a hook-and-loop-fastener, the laminate having a substrate and a textile web that is laminated to the substrate and that has a base structure knitted from threads and loop threads incorporated into the base structure by knitting and forming loops provided for attachment to hook-and-loop hooks, the improvement that the loop threads are made of a textured multifilament yarn.

2. The laminate according to claim 1, wherein the textured multifilament yarn of the loop threads has a linear density of between 35 dtex and 55 dtex and is formed from 11 to 25 filaments.

3. The laminate according to claim 1, wherein the textured multifilament yarn of the loop threads is made with a polymer base chosen from the group comprising polyethylene terephthalate, polyamide, polyactide and polypropylene.

4. The laminate according to claim 1, wherein the filaments of the textured multifilament yarn of the loop threads are twisted by an air jet.

5. The laminate according to claim 1, wherein the base structure is knitted from threads of a first base yarn and threads of a second base yarn.

6. The laminate according to claim 5, wherein the first base yarn and the second base yarn are made of polyamide.

7. The laminate according to claim 5, wherein the first base yarn is made of a monofilament yarn having a linear density of between 15 and 30 dtex and the second base yarn is made of a multifilament yarn having a linear density of between 35 and 55 dtex.

8. The laminate according to claim 1, wherein the textile web has a basis weight of between 15 and 45 grams per square meter.

9. The laminate according to claim 1, wherein the substrate is a film.

10. The laminate according to claim 9, wherein the film has a decorative printed pattern.

11. The laminate according to claim 9, wherein the thickness of the film is between 8 μm and 30 μm.

12. The laminate according to claim 1, wherein the substrate and the textile web are connected by an adhesive that does not cover their entire surface, and is instead arrayed in a pattern of adhesive surfaces and adhesive-free regions.

13. A method of making a laminate for a hook-and-loop fastener, the method comprising the steps of:
supplying a film as a substrate,
supplying a textile web that has a base structure knitted from threads and has loop threads incorporated into the base structure by knitting, the loop threads forming loops provided for attachment to hook-and-loop hooks and formed from a textured multifilament yarn,
bonding together the substrate and the textile web to form a material web by an adhesive arrayed in a pattern of adhesive surfaces and adhesive-free regions, and
separating individual pieces of the laminate from the material web.

14. The method according to claim 13, further comprising the step, once the material web has been produced, of:
bulking up the textured multifilament yarn of the loop threads using compressed air, brushes or some other mechanical treatment of the textile web.

15. The method according to claim 13, further comprising the steps of:
arranging the pieces of the laminate separated from the material web on a waist region of a diaper.
